(19) United States
(12) Patent Application Publication
(10) Pub. No.: US 2010/0029006 A1
(43) Pub. Date: Feb. 4, 2010

(54) MULTIPLEXED DIAGNOSTIC TEST FOR PRETERM LABOR

(76) Inventors: Kevin P. Rosenblatt, Houston, TX (US); Peter Bryant-Greenwood, Honolulu, HI (US)

Correspondence Address: FULBRIGHT & JAWORSKI L.L.P., 600 CONGRESS AVE., SUITE 2400, AUSTIN, TX, 78701, US

(21) Appl. No.: 12/535,187

(22) Filed: Aug. 4, 2009

Related U.S. Application Data
(60) Provisional application No. 61/085,968, filed on Aug. 4, 2008.

(51) Int. Cl. G01N 33/50

(52) U.S. Cl. 436/86

(57) ABSTRACT

Embodiments of the present invention relate to methods and devices for evaluating a pregnant subject and/or identifying risk of preterm labor in the pregnant subject. The inventors have observed that the initiation of preterm labor or its likelihood in a pregnant patient who has not yet completed 36 weeks gestation can be evaluated by measuring levels of one or more biomarker (marker) that is indicative of labor and/or preterm labor in a sample from the patient.

This application claims priority to U.S. Provisional Patent Application Ser. No. 61/085,968 filed Aug. 4, 2008, which is herein incorporated by reference in its entirety.

### BACKGROUND OF THE INVENTION

I. Field of the Invention

The present invention relates generally to the fields of medicine, obstetrics, and gynecology. More particularly, it concerns evaluating pregnant women for markers indicative of labor.

II. Background

Preterm labor is a complication in more than 12% of pregnancies in the United States each year, and represents both a serious health threat to mothers and babies. Preterm labor is a significant health care expense nationally. Despite its medical and fiscal impact, little is known about the underlying causes of preterm labor, and the applications of predictive or evaluative biomarker in reducing the incidence of preterm births has not been fully explored.

Preterm labor generally refers to active labor occurring before the end of the 36th week of pregnancy and it is characterized by cervical effacement, dilation, and increased uterine irritability (Weismiller, 1999). Studies have shown that women with a history of previous preterm delivery are at a greater risk of recurrence (Weismiller, 1999), and other maternal medical conditions have been variously associated with preterm labor, including chronic infections, a short cervical path, and poor nutrition.

Strategies to prevent preterm labor have focused on early diagnosis and on clinical biomarkers (Weismiller, 1999). Examples of these clinical biomarkers include monitoring cervical change, uterine contractions, bleeding, and changes in fetal behavioral states (Weismiller, 1999). Despite evaluation of such biomarkers however, women continue to enter into preterm labor with and without symptoms, resulting in fewer effective opportunities for slowing or halting the progression of labor. The subsequent expensive and sometimes medically complicated preterm birth can have lifelong consequences, as preterm babies are at much greater risk for cerebral palsy, mental retardation, and other health problems.

The underlying causes of preterm labor have yet to be elucidated and fully understood. Most studies focus on maternal and natal medical conditions subsequent to a preterm birth, offering little in the way of prognostic or diagnostic value. Consequently, there continues to be a widespread need for improved predictive and diagnostic tools in the treatment and prevention of preterm labor.

### SUMMARY OF THE INVENTION

The present invention relates to methods and devices for evaluating a pregnant subject and/or identifying risk of preterm labor in the pregnant subject. The inventors have observed that the initiation of preterm labor or its likelihood in a pregnant patient who has not yet completed 36 weeks gestation can be evaluated by measuring levels of one or more biomarkers (marker(s)) that is indicative of labor and/or preterm labor in a sample from the patient. Examples of labor biomarkers include serum retinol binding protein (RBP4, e.g., GenBank X00129 (GI:35896)), apolipoprotein C1 (ApoC1, e.g., GenBank AK312036 (GI:164698167) and X00570 (GI:28802)), pre-B-cell colony-enhancing factor (PBEF, e.g., GenBank BC106046 (GI:76779425)), interleukin 6 (IL-6, e.g., GenBank DQ894639 (GI:123995926)), interleukin 8 (IL-8, e.g., GenBank AK311874 (GI:164698005)), relaxin (e.g., GenBank DQ896468 (GI:123999916)), caspase recruitment domain containing protein CARD 12 (e.g., GenBank AY032589 (GI:13899172)), or ferritin (e.g, GenBank BC034419 (GI:21707935), DQ894293 (GI:123995234), EU831925.1 (GI:190691182)). All GenBank accession numbers are provided as an example and each is incorporated herein by reference as of the filing date of this application. Other variant sequences of these nucleotides can be identified in the GenBank database and are contemplated for use in and incorporated here by reference. In certain embodiments, the pregnant woman upon whose sample the methods of this invention are performed is at risk of preterm labor, or is suspected of being in labor or preterm labor.

Generally, the methods include binding a marker of interest or probe that recognizes a marker of interest to a substrate and detecting binding of the marker or the probe. Typically, the amount or levels of marker in a sample are quantified or semi-quantified. In certain aspects, determination of the presence or absence of a marker is sufficient. In a further aspect the method may comprise eluting, isolating, or otherwise dissociating the marker from the substrate; and analyzing the marker, for example by mass spectroscopy.

In certain aspects, methods of the invention can be adapted for lateral flow assays and devices supporting such. Lateral flow assays also known as immunochromatographic assays are typically carried out using a simple device intended to detect the presence (or absence) of a target analyte in sample. Most commonly these tests are used for medical diagnostics either for home testing, point of care testing, or laboratory use. Often produced in a dipstick format, these assays are a form of immunoassay in which the test sample flows along a solid substrate via capillary action. After the sample is applied to the test it encounters a colored or labeling reagent which mixes with the sample and transits the substrate encountering lines or zones which have been pretreated with an antibody or antigen or affinity reagent. Depending upon the analytes present in the sample the colored or labeling reagent can become bound at the test line or zone. Lateral flow assays can operate as either competitive or sandwich assays.

In still a further aspect, methods of the invention can be adapted for protein array assays and devices supporting such. Protein arrays or microarrays (also known as a biochip, or a proteinchip) are measurement devices used in biomedical applications to determine the presence and/or amount of proteins in biological samples, e.g. blood, urine, swabs, tissues scrappings, etc. Typically, a number of different capture agents, most frequently monoclonal antibodies, can be deposited on a chip surface (glass or silicon) in an array. This format is often also referred to as a microarray (a more general term for chip based biological measurement devices).

In further aspects, the invention provides that the measurement of levels of labor biomarker(s) using a blood, urine, amniotic fluid, cervical fluid, and/or a saliva sample from a pregnant subject. Certain aspects of the invention provides a method of measuring a labor biomarker comprising detecting biomarker proteins in a blood sample by contacting the sample with one or more specific antibodies or binding agents. In certain aspects, affinity reagents are configured on a substrate, for example as a protein array. In yet another aspect, methods for measuring the level of nucleic acids encoding for labor biomarkers can be used (e.g., nucleic acid arrays). In some aspects, the invention provides for the detection of nucleic acids by amplification, hybridization, or a combination thereof.

In certain aspects, the invention provides a method for predicting or assessing the initiation of labor or preterm labor. In certain aspects, levels of biomarker in a sample from a pregnant subject are compared to the levels of those biomarkers in a predetermined standard, which standard may be a sample containing a range of biomarker levels which correspond to those expected in the population of pregnant women who are not at risk of beginning preterm labor.

In particular aspects, the invention provides a method for identifying preterm labor in progress and for differentiating early stages of preterm labor from active preterm labor. In some aspects, levels of a biomarker in a sample from a pregnant subject are compared to the levels of those biomarkers in a predetermined standard. A method is also provided for measuring levels of preterm labor biomarkers at multiple points in time as a means of monitoring the progression of labor or preterm labor. In some embodiments, the predetermined standard is a range of biomarker levels representative of a pregnant subject who is less than 36 weeks gestation and not in an early or active stage of preterm labor, or representative of a pregnant subject that is in active labor after 36 weeks of gestation. Gestation is defined as the carrying of an embryo or fetus inside a female. The time interval of a gestation plus 2 weeks is called gestation period, and the length of time plus 2 weeks that the offspring have spent developing in the uterus is called gestational age. The extra 2 weeks is because typically gestational age is counted starting from the last menstrual period (LMP), rather than actual conception.

A more effective method for identifying individuals in and at risk of starting preterm labor can provide more options for effective medical treatment, rendering more likely the prevention of preterm labor or the cessation or treatment of preterm labor that is in progress. As such, the present invention includes methods for aiding in the prevention or inhibition or amelioration of preterm labor and for assessing treatment effectiveness in a subject who has been determined to be in preterm labor or who is at risk of beginning preterm labor. These methods comprise one or more of the following steps: (1) the detection and/or measurement of one or more labor biomarker selected from ApoC1, PBEF, RBP4, IL-6, IL-8, relaxin, CARD 12, and/or ferritin at one or more time points, (2) a comparison of the levels of biomarkers measured in the subject to the levels of preterm labor biomarkers in one or more of a predetermined standard, and (3) administration of medically appropriate preventive or retardant measures to prevent or inhibit preterm labor. In certain aspects these markers can be measured in conjunction with other known markers of labor.

In certain embodiments, the invention provides for a method of determining and/or predicting preterm labor in a pregnant patient with a sensitivity of at least about, at most about, or about 40, 50, 60, 70, 75, 80, 85, 90, 91, 92, 93, 94, 95, 96% including all values and ranges there between. The invention can also provide a positive predictive value (PPV) of at least about, at most about, or about 40, 50, 60, 70, 75, 80, 85, 90, 91, 92, 93, 94, 95, 96% including all values and ranges there between. The invention can also provide a negative predictive value (NPV) of at least about, at most about, or about 40, 50, 60, 70, 75, 80, 85, 90, 91, 92, 93, 94, 95, 96% including all values and ranges there between.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Serum samples were thawed, added to a IMAC interaction protein chip, and analyzed on the Protein Biology System 2 SELDI-TOF mass spectrometer (Ciphergen Biosystems, Freemont, Calif., USA). Mass resolution (defined as m/Δm) is routinely achieved below 400. Mass accuracy is assessed daily through the use of angiotensin peptide calibrations. A mass accuracy of 0.1% was achieved with this system. Peptides and proteins below the 20 000 mass/charge (M/Z) range were ionized with -cyano-4-hydroxycinnamicacid as a matrix, which is most effective for the detection of proteins and peptides in this mass range. The chips were analyzed manually under the following settings: laser intensity 240, detector sensitivity 10, mass focus 6000, position 50, molecular mass range 0-20000 Da, and a 50-shot average per sample. Data were collected without filters and were later used for analyses. Labor and non-labor samples and controls were run concurrently, intermingled on the same chip; the operators were blinded to sample identity.

FIG. 2. In a full dataset labor was discriminated from false labor in the term and pre-term period with 82% sensitivity and 76% specificity using 25 data points. One particular peak at 5908.6 m/z was able to model with an accuracy of 60% with 12 false positives and 10 false negatives. Based on this single value, it was clear that there was poor separation of the labor and non-labor groups when the average peak amplitude of 5908.6 m/z plus are compared using the entire population. The bold line represents a plus one standard deviation from the mean.

FIG. 3. When corrected for maternal infection, the mean amplitude of 5908.6 value for the non-labor group dropped from 16.1 to 12.4. The algorithm was re-trained to assess for labor (delivery within 24 hours) or intrauterine infection in the Asian/Pacific Islander population. The result increased the predicted accuracy of the 5908.6 m/z value to 96%.

FIG. 4. Lastly, two other m/z values from the original 25 data points were used in the final model (2953.4 and 4125.6). This further improved diagnostic accuracy to 98%.

FIGS. 5A-5D. Discovery and Validation of Labor and PTB Markers. (FIG. 5A) The figure displays a mass spectral summary of average intensities for labor and non-labor. The Y-axis denotes the Relative Intensities of the spectral peaks; the X-axis specifies the mass-to-charge ratios (m/z) of the different protein species in Daltons. Note that at least four different peaks were found, using a high-resolution MALDI approach, which discriminated between labor and non-labor. (FIG. 5B) The figure gives an example of our high-throughput, protein microrray analysis for one of our markers. Three separate dilutions (1:30, 1:90, and 1:270) for 318 individual patient samples are shown. Four microarray spotting pins arrayed patient blood samples in 4 blocks (labeled 1-4). Quantum-dot labeled antibodies were used to detect the relevant markers (here Marker #1=PTB#1). The scale represents relative intensities over a gradient set with blue at the lowest level and red at the highest. The different blocks were normalized within and between them to correct for “position effects” (changes in fluorescent intensities due to differences in microarray spotting, or changes in intensity due to variations in the slides, or unequal staining of the spots secondary to unequal application of the quantum dots) on the array. (FIGS. 5C and 5D) ROC Curves and their integral (area under the curves) values are shown for Labor (L) and Not-in-Labor (NIL, aka Non-Labor). A curve lying along the diagonal line through the origin would indicate markers without discriminative power.

### DETAILED DESCRIPTION OF THE INVENTION

Human pregnancy can be divided into three trimesters, each three months long. The first trimester is from 0 to 12 weeks, the second from 12 to 28 weeks and the third begins at about 28 weeks. In humans, birth normally occurs at a gestational age of 37 to 42 weeks. Childbirth occurring before 37 weeks of gestation is considered preterm, childbirth after 25 weeks is usually considered “viable.” Preterm and low birth weight babies are a contributing factor to infant death, particularly those that die within twenty-four hours of birth.

### I. Labor, Premature Birth, and Preterm Risks

Labor is a physiologic process during which the products of conception (i.e., the fetus, membranes, umbilical cord, and placenta) are expelled outside of the uterus. Labor is achieved with changes in the connective tissue and with gradual effacement and dilatation of the uterine cervix as a result of rhythmic uterine contractions of sufficient frequency, intensity, and duration (American College of Obstetricians and Gynecologists [ACOG], 2003; Norwitz, 2003).

Typically, labor is a clinical diagnosis. The onset of labor is defined as regular, painful uterine contractions resulting in progressive cervical effacement and dilatation. Cervical dilatation in the absence of uterine contraction suggests cervical incompetence, whereas uterine contraction without cervical change does not meet the definition of labor.

Obstetricians have divided labor into 3 stages that delineate milestones in a continuous process. The first stage begins with regular uterine contractions and ends with complete cervical dilatation at approximately 10 cm. In his landmark studies of 500 nulliparas (women giving birth for the first time), Friedman (1955) subdivided the first stage into an early latent phase and an ensuing active phase. The latent phase describes the period between the onset of labor and when the rate of cervical dilatation changes most rapidly, usually at about 3-4 cm of cervical dilatation. The active phase heralds a period of increased rapidity of cervical dilation and ends with complete cervical dilation of 10 cm. According to Friedman, the active phase is further divided into an acceleration phase, a phase of maximum slope, and a deceleration phase.

Characteristics of the average cervical dilatation curve is known as the Friedman curve, and a series of definitions of labor protraction and arrest were subsequently established (Friedman, 1961a and 1961b). However, subsequent data suggest that the rate of cervical dilatation is slower and the progression of labor may be significantly different from that shown on the Friedman curve (Kilpatrick, 1989; Albers, 1996; Zhang, 2002).

The second stage begins with complete cervical dilatation and ends with the delivery of the fetus. The ACOG has suggested that a prolonged second stage of labor should be considered when the second stage exceeds 3 hours if regional anesthesia is administered or 2 hours in the absence of regional anesthesia in nulliparas. In multiparous women, such a diagnosis can be made if the second stage of labor exceeds 2 hours with regional anesthesia or 1 hour without it (ACOG, 2003).

Studies performed to examine perinatal outcomes associated with a prolonged second stage of labor revealed increased risks of surgical deliveries and maternal morbidities but no differences in neonatal outcomes (Menticoglou 1995; Janni, 2002; Cheng, 2003; Myles, 2003). Maternal risk factors associated with a prolonged second stage include nulliparity, maternal weight and/or weight gain, use of regional anesthesia, induction of labor, fetal occiput in a posterior position, and increased birthweight (Cheng, 2003; Myles 2003; O'Connell, 2003; Senécal, 2005).

The third stage of labor lasts from the delivery of the fetus until the delivery of the placenta and fetal membranes. Although delivery of the placenta requires less than 10 minutes, the duration of the third stage of labor may last as long as 30 minutes before active intervention is commonly considered (Norwitz, 2003).

Preterm labor is a major factor in perinatal morbidity and mortality in the United States, resulting in approximately 500,000 high-risk births every year at an annual average cost of 26 billion USD. Preterm labor, defined as the onset of active labor prior to the 37^{th} week of gestation, most commonly results in preterm birth, which, despite advances in the medical treatment of preterm labor in terms of delaying labor and preparing infants for premature birth, is still the number one cause of neonatal mortality in the U.S. (National Center for Health Statistics, 2004). In addition, it has recently been reported that 79% of preterm infants that survive birth will face one or more complications (Willson, 2003) including respiratory distress syndrome, bronchopulmonary dysplasia, pneumonia, apnea, bradycardia, jaundice, intraventricular hemorrhage, patent ductus arteriosus, hypothermia, anemia, retinopathy, necrotizing enterocolitis, sepsis, and gastrointestinal dysfunction (National Center for Health Statistics, 2004). While the health and survival rates of premature infants have improved in recent years, the actual rate of premature births remains consistent, in part because so little is known about the causes of preterm labor and how to identify those at risk early enough to initiate effective medical intervention (Weismiller, 1999).

Preterm labor may or may not be associated with vaginal bleeding or rupture of membranes. A number of medical conditions and other factors have been correlated with the onset of preterm labor, including, but not limited to, infection (e.g., bacterial vaginosis [BV], sexually transmitted diseases [STDs], urinary tract infections, chorioamnionitis), uterine distention (e.g., multiple gestation, polyhydramnios), uterine distortion (e.g., mullerian duct abnormalities, fibroid uterus), compromised structural support of the cervix (e.g., incompetent cervix, previous cone biopsy or loop electrosurgical excision procedure [LEEP]), abruptio placentae, uteroplacental insufficiency (e.g., hypertension, insulin-dependent diabetes, drug abuse, smoking, alcohol consumption), stress either indirectly by associated risk behaviors or by direct mechanisms including fetal stress.

In certain aspects, the current invention provides additional predictive and diagnostic tools for assessment of labor and for the treatment of preterm labor giving health care providers and patients more timely information as well as more effective medical options for avoiding preterm deliveries and the attendant health disadvantages.

### II. Biomarkers of Labor and/or Preterm Labor

The term “labor biomarker”, “preterm labor biomarker” or “PTL biomarker” includes a biomarker/marker associated with labor or preterm labor, in certain aspects, a polypeptide biomarker associated with labor or preterm labor. Typically, a biomarker is selected from, but not limited to, the group consisting of serum retinol binding protein (RBP4), apolipoprotein C1 (ApoC1), pre-B-cell colony-enhancing factor (PBEF), interleukin 6 (IL-6), interleukin 8 (IL-8), relaxin, caspase recruitment domain containing protein CARD 12, and ferritin. The term includes wildtype polypeptides and polypeptide isoforms, as well as polypeptide variants, chimeric polypeptides, complexes, fragments, precursors, modified forms, and derivatives of the biomarkers. The term also includes a biomarker associated with preterm labor identified using a method of the invention, in particular a biomarker associated with preterm delivery less than 96, 72, 48, or 24 hours from clinical presentation including all values and ranges there between; preterm delivery less than 25, 26, 27, 28, 29, 30, 31, 32, 33, 34, 35 or 36 weeks gestation including all values and ranges there between; and preterm delivery less than 25, 26, 27, 28, 29, 30, 31, 32, 33, 34, 35, 36, or 37 weeks gestation including all values and ranges there between.

A “wildtype polypeptide” comprises a polypeptide having the same amino acid sequence of a polypeptide derived from nature. Such wildtype polypeptides can be isolated from nature or can be produced by recombinant or synthetic means. The term specifically encompasses naturally occurring truncated or secreted forms of a polypeptide, polypeptide variants including naturally occurring variant forms (e.g., alternatively spliced forms or splice variants), and naturally occurring allelic variants.

The term “polypeptide variant” means a polypeptide having at least about 70-80%, at least about 85%, at least about 90%, at least about 95% amino acid sequence identity with a wildtype polypeptide. Polypeptide variants typically have at least 70-80%, 85%, 90%, 95% amino acid sequence identity to the sequences of the biomarkers identified for use in the current invention. Such variants include, for instance, polypeptides wherein one or more amino acid residues are added to, or deleted from, the N- or C-terminus of the full-length or mature sequences of the polypeptide, including variants from other species. In certain aspects the term variant can exclude a wildtype polypeptide. Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length.

The invention also includes polypeptides that are substantially identical to the sequences of a PTL Biomarker, in particular a preterm labor biomarker, more particularly a biomarker expressed by a polynucleotide sequence which is at least about 45%, preferably 50%, 55%, 60%, 65%, 70%, 75%, 80%, 85%, 90%, 95%,97%, 98%, or 99% sequence identity), and in particular polypeptides that retain the immunogenic activity of the corresponding wildtype polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410,1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

A naturally occurring variant may contain conservative amino acid substitutions from the native polypeptide sequence or it may contain a substitution of an amino acid from a corresponding position in a polypeptide homolog or include a homolog of the marker, for example, a horse, cow, mouse, or rat polypeptide.

A modified form of a polypeptide referenced herein includes modified forms of the polypeptides and derivatives of the polypeptides, including but not limited to glycosylated, phosphorylated, acetylated, methylated, or lipidated forms of the polypeptides.

The invention provides assessment of biomarkers that can correlate with labor and/or preterm labor by clustering analysis. A subset of these biomarkers are identified for detection, assessment, diagnosis, prevention, and therapy of preterm labor. The invention also provides a method of using these biomarkers to distinguish threatened preterm labor that progresses to delivery from pregnancies that continue to term.

The biomarkers that can be used to identify labor, distinguish imminent preterm labor from ongoing preterm labor, monitor the progress or identify the stages of labor or preterm labor, and assess the effectiveness of treatment to slow preterm labor or prevent preterm delivery. In one aspect, the invention provides a method for classifying a patient sample as indicative of preterm labor comprising detecting a difference in the levels of one or more biomarkers from the sample relative to the levels of the corresponding biomarkers in one or more normal control samples or standards. By way of non-limiting example, a normal control sample or standard can comprise a serum sample derived from one or more patient who is not in labor or preterm labor, a serum sample derived from one or more patient who is not in preterm labor, a serum sample derived from one or more patients who are not predisposed to preterm labor or delivery, a serum sample derived from one or more patients who are confirmed to be in a specific stage of labor or preterm labor.

Any of the biomarkers provided herein may be used alone or with other biomarkers of labor or preterm labor, or with biomarkers for other phenotypes or conditions. Other biomarkers include thrombospondin 2, decorin, matrix metalloproteinase-12, matrix metalloproteinase-8, matrix metalloproteinase-1, matrix metalloproteinase-2, matrix metalloproteinase-3, matrix metalloproteinase-9, hyaluronidase, lumican, fibromodulin, keratocan, PRELP biglcan, mimecan, serglycin, perlecan, agrin, versican, link proteins, CD44, TSG-6, bikunin, inter alpha trypsin inhibitor, a hyaluranan synthase, hyaluronic acid, elastin, fibulins, fibrillins, tenascin X, heparan sulfate, keratan sulfate, chondroitin sulfate, dermatan sulfate, heparanase, lysyl oxidase, type V collagen, type VI collagen, type XII collagen, type XIV collagen, type XVIII collagen, type XX collagen, calreticulin, CD47, syndecans, glypicans, lipoprotein receptor-like protein (LRP), an ADAM protease, an ADAMTS protease, E-selectin, L-selectin, platelet factor 4, transforming growth factor beta 1, 2 and 3 (TGF-.beta.), epidermal growth factor (EGF), keratinocyte growth factor (KGF), vascular endothelial growth factor (VEGF), hepatocyte growth factor (HGF), fibroblast growth factors (FGF) 1 and 2, and platelet derived growth factor (PDGF), and combinations thereof.

### III. Subject Samples

In describing the current invention, the terms “subject”, “individual” or “patient” refer to a female mammal, particularly a female human. A subject, individual or patient may be afflicted with or suspected of having or being pre-disposed to preterm labor. The present invention may be particularly useful for determining preterm labor development potential in at-risk patients suffering from particular preterm labor predisposing conditions. Preterm labor predisposing conditions include without limitation a previous history of preterm delivery, previous history of a second-trimester abortion, uterine factors such as uterine volume increase, uterine anomalies, trauma, and/or infection.

The terms “sample”, “biological sample”, and the like mean a material known or suspected of containing one or more labor biomarkers or other markers. A test sample can be used directly as obtained from the source or following a pretreatment to modify the character of the sample. A sample can be derived from any biological source, such as tissues, extracts, or cell cultures, including cells, cell lysates, and physiological fluids, such as, for example, whole blood, plasma, serum, cervical fluid or swabs, saliva, urine, milk, amniotic fluid, peritoneal fluid, and the like.

The sample can be obtained from mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like.

In certain aspects of the invention, the sample is a blood sample from a subject, in particular a serum sample from a subject, and more particularly a serum sample from a pregnant female subject. In one embodiment, a serum sample from a pregnant female subject is analyzed for levels of labor biomarkers within 1, 2, 3, 4, 5, or more hours of its collection. In another embodiment, a serum sample has been frozen and thawed prior to analysis.

### IV. Polypeptides and Polypeptide Detection Methods

In certain embodiments, the present invention provides a method for assessing labor and/or diagnosing potential for preterm labor, imminent initiation of preterm labor or ongoing preterm labor, the method comprising at least the measurement in a sample of the levels of one or more biomarker polypeptides by contacting the sample with one or more binding agents that specifically bind to biomarkers or parts thereof; detecting the bound biomarker and/or biomarker level for the sample, and comparing the biomarker levels in the patient sample to the corresponding biomarker levels in a normal sample or standard. In some embodiments of the invention, one or more biomarker binding and/or detection agents are configured on an array and/or operatively coupled to a substrate.

“Binding agent” refers to a substance such as a surface, particle, polypeptide or antibody that binds to one or more PTL Biomarker. A substance “specifically binds” to one or more Biomarker if it reacts at a detectable level with one or more biomarker, and does not react detectably with peptides or other substances containing an unrelated or different amino acid sequence or structure. Binding properties may be assessed using an immunoassay such as an ELISA, which may be readily performed by those skilled in the art (see for example, Newton et al., Develop. Dynamics 197: 1-13, 1993).

Binding agents may be used for a variety of diagnostic and assay applications. There are a variety of assay formats known to the skilled artisan for using a binding agent to detect a target molecule in a sample (for example, see Harlow and Lane, Antibodies: A Laboratory Manual, Cold Spring Harbor Laboratory, 1988). In general, the presence or absence of preterm labor or stage or type of preterm labor in a subject may be determined by (a) contacting a sample from the subject with a binding agent; (b) detecting in the sample a level of polypeptide(s) that binds to the binding agent; and (c) comparing the level of polypeptide(s) with a predetermined standard or cut-off value. The polypeptides detected are typically those associated with labor and/or pre-term labor.

In particular embodiments of the invention, the binding agent is an antibody. Antibodies specifically reactive with one or more biomarkers, or derivatives, such as enzyme conjugates or labeled derivatives, may be used to detect one or more biomarkers in various samples (e.g., biological materials). They may be used as diagnostic or prognostic reagents and they may be used to detect abnormalities in the level of expression or presence of one or more biomarker, or abnormalities in the structure, and/or temporal, tissue, cellular, or subcellular location of one or more biomarker. An abnormality in one or more biomarker is typically indicative of physiologic condition, such as labor or preterm labor. Antibodies may be used to screen potentially therapeutic compounds to determine their effects on preterm labor as indicated by levels of one or more biomarkers. In vitro immunoassays may also be used to assess or monitor the efficacy of particular therapies.

Antibodies used as binding agents in a method of the invention include, but are not limited to antibodies which specifically bind a biomarker polypeptide selected from the group consisting of serum retinol binding protein (RBP4), apolipoprotein C1 (ApoC1), pre-B-cell colony-enhancing factor (PBEF), interleukin 6 (IL-6), interleukin 8 (IL-8), relaxin, caspase recruitment domain containing protein CARD 12, and ferritin.

Antibodies for use in the present invention include but are not limited to monoclonal or polyclonal antibodies, immunologically active fragments (e.g., a Fab or (Fab)₂ fragments), antibody heavy chains, humanized antibodies, antibody light chains, genetically engineered single chain Fv molecules (U.S. Pat. No. 4,946,778, which is incorporated herein by reference in its entirety), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments, and chimeras, may be prepared using methods known to those skilled in the art. Isolated native or recombinant biomarkers may be utilized to prepare such antibodies. See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for a PTL Biomarker may also be obtained from scientific or commercial sources. In certain aspects antibodies that specifically bind biomarkers including any combination or permutation of 1, 2, 3, 4, 5, 6, 7, or 8 of serum retinol binding protein (RBP4), apolipoprotein C1 (ApoC1), pre-B-cell colony-enhancing factor (PBEF), interleukin 6 (IL-6), interleukin 8 (IL-8), relaxin, caspase recruitment domain containing protein CARD 12, and ferritin can be configured on a substrate and form a protein chip, for example. In other embodiments the biomarker may interact with a particular characteristic of a surface via hydrophobicity, hydrophilicity, charge, etc, thus specific binding of a biomarker is not required. In some embodiments, the protein chip is an IMAC interaction chip.

“Micro-array” and “array,” refer to protein or peptide arrays or nucleic acid or nucleotide arrays that can be used to detect biomolecules associated with labor or preterm labor, for instance to measure biomarker polypeptide levels in a serum sample. A variety of arrays are made in research and manufacturing facilities worldwide, some of which are available commercially.

Polypeptides may, but need not, be digested into peptides, in particular using proteolytic enzymes such as trypsin, pepsin, subtilisin, and proteinase. For example, polypeptides may be treated with trypsin which cleaves at the sites of lysine and arginine to provide peptides with a length of from about 5 to 50 amino acids. Such peptides may be particularly appropriate for mass spectrometry analysis, especially electrospray ionization mass spectrometry. Chemical reagents including cyanogen bromide may also be utilized to digest proteins.

In an aspect, the invention provides a diagnostic method for monitoring or diagnosing preterm labor in a subject by quantitating one or more biomarkers in a biological sample from the subject by reacting the sample with antibodies specific for one or more biomarkers. The antibodies can be directly or indirectly labeled with detectable substances. In a particular embodiment of the invention, biomarkers are quantitated or measured.

The invention also relates to a method of characterizing a sample, such as a serum sample from a pregnant patient, by detecting or quantitating in the sample one or more biomarker polypeptides in or extracted from the sample. In one aspect, the method comprises assaying for levels of one or more biomarkers in the sample that differ from a subject not in PTL or in labor. In other aspects, the differential profiles of biomarkers can be assayed in samples by contacting the sample with a binding agent configured on an array and/or a substrate (alternatively contacting a surface that differentially binds polypeptides) and subjecting the sample to mass spectrometry analysis. In some embodiments, the mass spectrometry analysis is a SELDI-TOF analysis.

“SELDI-TOF” refers to a specialized kind of mass spectrometry technique for analyzing biomolecules. In general, mass spectrometry analysis of a biomolecular sample involves the chemical fragmentation of the sample into individual ions, the measurement of both charge and mass of the ions by passing the particles through electric and magnetic fields, and the determination of the mass to charge ratio for the individual ions in the sample. A particular type of instrument used in mass spectrometry analysis is a time-of-flight (TOF) spectrometer, in which an electrical field only is used and the time that ions in an ionized sample take to reach the detector of the instrument provides the mass to charge ratio of the individual ions in the sample. Surface-enhanced laser desorption/ionization, or SELDI, is an ionization technique often used with a TOF spectrometer and involves an added affinity step to the basic mass spectrometry technique. SELDI is therefore particularly useful for the analysis of complex polypeptide and peptide mixtures sometimes encountered in blood and other kinds of clinical samples. As a nonlimiting example of the application of SELDI to the present invention, an IMAC surface array chip is (1) contacted with a sample containing biomarkers of interest, (2) washed to remove nonspecific interactions, (3) contacted with an appropriate matrix material for optimized ionization, and (4) analyzed using a TOF mass spectrometer.

Mass spectrometers which may be used in accordance with a method of the invention include without limitation a Matrix-Assisted Laser Desorption/Ionization Time-of-Flight Mass Spectrometer (“MALDI-TOF”) (e.g., from PerSeptive Biosystems, Framingham, Mass.); an Electrospray Ionization (“ESI”) ion trap spectrometer, (e.g., from Finnigan MAT, San Jose, Calif.), an ESI quadrupole mass spectrometer (e.g., from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g., from Ciphergen Biosystems Inc.). In certain embodiments, a SELDI-TOF mass spectrometer is used to measure levels of biomarkers in samples.

One embodiment of the invention comprises the following steps (a) incubating a biological sample with first antibodies specific for one or more biomarkers that are directly or indirectly labeled with a detectable substance, and second antibodies specific for one or more biomarkers which are immobilized; (b) detecting the detectable substance thereby quantitating or detecting biomarkers in the biological sample; and (c) comparing the quantitated or detected biomarkers with a standard or reference.

The standard may correspond to levels quantitated for samples from control subjects without preterm labor (normal), with a different stage of preterm labor, or from other samples of the subject. In an embodiment, increased levels of biomarkers as compared to the standard may be indicative of labor or preterm labor. In another embodiment, lower levels of biomarkers as compared to the standard may be indicative of preterm labor.

Embodiments of the methods of the invention involve (a) reacting a biological sample from a subject with antibodies specific for one or more biomarkers that are directly or indirectly labeled with an enzyme; (b) adding a substrate for the enzyme wherein the substrate is selected so that the substrate, or a reaction product of the enzyme and substrate forms fluorescent complexes; (c) quantitating one or more biomarkers in the sample by measuring fluorescence of the fluorescent complexes; and (d) comparing the levels obtained for other samples from the subject patient, or control subjects.

Antibodies or binding agents may be used in any known immunoassays that rely on the binding interaction between antigenic determinants of one or more biomarker and the antibodies. Immunoassay procedures for detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more PTL biomarker in a sample may be accomplished by competitive or non-competitive immunoassay procedures in either a direct or indirect format. Examples of immunoassays are radioimmunoassays (RIA), enzyme immunoassays (e.g., ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

In certain aspects of the invention, an immunoassay for detecting more than one biomarker in a biological sample comprises contacting binding agents that specifically bind to biomarkers in the sample under conditions that allow the formation of first complexes comprising a binding agent and biomarkers and determining the presence or amount of the complexes as a measure of the amount of biomarkers contained in the sample. In a particular embodiment, the binding agents are labeled differently or are capable of binding to different labels.

Binding agents (e.g antibodies) may be used in immunohistochemical analyses, for example, at the cellular and sub-subcellular level, to detect one or more biomarkers, to localize them to particular cells and tissues, and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978).

Antibodies specific for one or more biomarker may be labeled with a detectable substance and localized in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent biomarker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualized by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more biomarkers. By way of example, if the antibody having specificity against one or more biomarkers is a rabbit IgG antibody, the second antibody may be goat anti-rabbit gamma-globulin labeled with a detectable substance as described herein.

Methods for conjugating or labeling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labeling the antibodies with enzyme or ligand binding partner).

Cytochemical techniques known in the art for localizing antigens using light and electron microscopy may be used to detect one or more PTL biomarkers. Generally, antibodies may be labeled with detectable substances and one or more PTL biomarkers may be localized in tissues and cells based upon the presence of the detectable substances.

In the context of the methods of the invention, the sample, binding agents (e.g. antibodies specific for one or more biomarkers), or one or more biomarkers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a biomarker may be immobilized using sheep anti-mouse IgG Fc fragment specific antibody coated on the carrier or support.

One or more biomarker antibodies may also be indirectly labeled with an enzyme using ligand binding pairs. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for biomarker antibody is labeled with an enzyme.

In certain embodiments, protein measurement and detection methods are used to measure increased or decreased levels of a biomarker in a sample from a pregnant subject. In certain aspects, the subject is suspected of being in preterm labor or at risk of entering into preterm labor. Examples of protein measurement methods include without limitation enzyme linked immunosorbent assay (ELISA), radioimmunoassay (RIA), immunoradiometric assay, immunohistochemistry, fluoroimmunoassay, chemiluminescent assay, bioluminescent assay, and Western blot to mention a few. The steps of various useful immunodetection methods have been described in the scientific literature, such as, e.g., Doolittle and Ben-Zeev, 1999; Gulbis and Galand, 1993; De Jager et al., 1993; Nakamura et al., 1987, each incorporated herein by reference.

A. Protein Array Technology

Protein array technology allows high-throughput screening for polypeptides, gene expression, and molecular interactions. In one aspect protein arrays enable the translation of gene expression patterns of normal and diseased tissues into protein product catalog. Protein function, such as enzyme activity, antibody specificity, and other ligand-receptor interactions and binding of nucleic acids or small molecules can be analyzed.

1. Protein Biochip Assays

These arrays, which contain thousands of different proteins or antibodies spotted onto glass slides or interactive surfaces, or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent biomarkers or other methods of detection revealing the spots that have captured these proteins, protein microarrays are being used as powerful tools in high-throughput proteomics and drug discovery.

Glass slides are still widely used, since they are inexpensive and compatible with standard microarrayer and detection equipment. However, their limitations include multiple-based reactions, high evaporation rates, and possible cross-contamination.

Matrix slides offer a number of advantages, such as reduced evaporation and no possibility of cross-contamination, but they are expensive. Nanochips for proteomics have the same advantages, in addition to reduced cost and the capability of multiple-component reactions.

A well-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system enables biomarker pattern recognition analysis to be done. This system allows one to address important clinical questions by investigating the proteome from a range of crude clinical samples (i.e., serum, laser capture microdissected cells, biopsies, tissue, and urine). The system also utilizes biomarker pattern software that automates pattern recognition-based statistical analysis methods to correlate protein expression patterns from clinical samples with disease phenotypes. Its robotics system accessory automates sample processing, allowing hundreds of samples to be run per week and enabling a sufficient number of samples to be run, which provides high statistical confidence in comprehensive studies for biomarker discovery and validation.

2. Microfluidic Chip-Based Immunoassays

Microfluidics is an important innovation in biochip technology. Since microfluidic chips can be combined with mass spectrometric analysis, a microfluidic device has been devised in which an electrospray interface to a mass spectrometer is integrated with a capillary electrophoresis channel, an injector, and a protein digestion bed on a monolithic substrate (Wang et al., 2000). This chip thus provides a convenient platform for automated sample processing in proteomics applications.

These chips can also analyze expression levels of serum proteins with detection limits comparable to commercial enzyme-linked immunosorbent assays, with the advantage that the required volume sample is markedly lower compared with conventional technologies.

Biosite (San Diego) manufactures the Triage protein chip that simultaneously measures 100 different proteins by immunoassays. The Triage protein chip immunoassays are performed in a microfluidic plastic chip, and the results are achieved in 15 minutes with picomolar sensitivities. Microfluidic fluid flow is controlled in the protein chip by the surface architecture and surface hydrophobicity in the microcapillaries. The immunoassays utilize high-affinity antibodies and a near-infrared fluorescent label, which is read by a fluorometer.

3. Nanoscale Protein Analysis

Most current protocols including protein purification and automated identification schemes yield low recoveries that limit the overall process in terms of sensitivity and speed. Such low protein yields and proteins that can only be isolated from limited source material (e.g., biopsies) can be subjected to nanoscale protein analysis: a nanocapture of specific proteins and complexes, and optimization of all subsequent sample-handling steps, leading to a mass analysis of peptide fragments. This focused approach, also termed targeted proteomics, involves examining subsets of the proteome (e.g., those proteins that are specifically modified, bind to a particular binding agent or sequence, or exist as members of higher-order complexes or any combination thereof). This approach can be used to measure PTL biomarkers in serum. The detection technique called multiphoton detection, by Biotrace Inc. (Cincinnati), can quantify subzeptomole amounts of proteins and can be used for diagnostic proteomics, particularly for cytokines and other low-abundance proteins.

B. Immunobinding Assays

In general, the immunobinding methods involve measurement of the formation of immunocomplexes. In these instances, the antibody binds and/or removes the biomarker from a sample. The antibody will preferably be linked to a solid support, such as in the form of a planar substrate or column matrix, and the sample suspected of containing the a biomarker will be applied to the immobilized antibody. The unwanted components will be washed from the binding surface, leaving the biomarker immunocomplexed to the immobilized antibody.

The immunobinding methods also include methods for detecting and quantifying the amount of a biomarker component in a sample and the detection and quantification of any immune complexes formed during the binding process. Here, one would obtain a sample suspected of containing a biomarker, and contact the sample with an antibody against a biomarker, and then detect and quantify the amount of immune complexes formed under the specific conditions.

In terms of detection, the biological sample analyzed may be any sample that is suspected of containing a biomarker, such as, for example, a biological fluid (blood, urine, saliva, etc.), a tissue swab, scrap, section or specimen, a homogenized tissue extract, a cell, an organelle, separated and/or purified forms of any of the above compositions.

Contacting the chosen biological sample with the antibody under effective conditions and for a period of time sufficient to allow the formation of immune complexes (primary immune complexes) is generally a matter of simply adding the antibody composition to the sample or the sample to the antibody composition, and incubating the mixture for a period of time long enough for the antibodies to form immune complexes with, i.e., to bind to, biomarkers present. After this time, the sample-antibody composition, such as a protein array, a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or biomarker, such as any of those radioactive, fluorescent, biological, and/or enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

Any antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification.

One method of immunodetection uses two different antibodies. A first step biotinylated, monoclonal or polyclonal antibody is used to detect the target(s), and a second step antibody is then used to detect the biotin attached to the complexed biotin. In that method the sample to be tested is first incubated in a solution containing the first step antibody. If the target is present, some of the antibody binds to the target to form a biotinylated antibody/target complex. The antibody/target complex is then amplified by incubation in successive solutions of streptavidin (or avidin), biotinylated DNA, and/or complementary biotinylated DNA, with each step adding additional biotin sites to the antibody/target complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/target complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

1. Western Blot Analysis

Western blot analysis is an established technique that is commonly employed for analyzing and identifying proteins. The proteins are first separated by electrophoresis in polyacrylamide gel, then transferred (“blotted”) onto a nitrocellulose membrane or treated paper, where they bind in the same pattern as they formed in the gel. The antigen is overlaid first with antibody, then with anti-immunoglobulin or protein A labeled with a radioisotope, fluorescent dye, or enzyme. One of ordinary skill in the art would be familiar with this commonly used technique for quantifying protein in a sample.

2. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used. One of ordinary skill in the art would be familiar with use of ELISAs and other immunohistochemical assays.

### V. Nucleic Acid Detection Methods

Certain embodiments of the present invention concerns the preparation and use of nucleic acids or nucleic acid arrays. A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect mRNA or genes or nucleic acid representative of genes associated with PTLs of the invention.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; W00138580; WO 0168255; WO 03020898; WO 03040410; WO 03053586; WO 03087297; WO 03091426; W003100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or nucleic acid targets. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

A. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a preterm labor sample labeled with Cy3, and normal labor sample labeled with Cy5. Differences between the samples for particular nucleic acids corresponding to probes on the array can be readily ascertained and quantified.

B. Differential Expression Analyses

Arrays of the invention can be used to detect differences between two samples. Specifically contemplated applications include identifying and/or quantifying differences between nucleic acid or gene expression from a sample that is normal and from a sample that is not normal, between a disease or condition and a cell not exhibiting such a disease or condition, or between two differently treated samples. Also, nucleic acid or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition (e.g., preterm labor), or one believed to be not normal with respect to that disease or condition. Phenotypic traits include symptoms of, or susceptibility to, a disease or condition of which a component is or may or may not be genetic.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

In certain embodiments, nucleic acid and/or expression profiles may be generated to evaluate and correlate those profiles with pharmacokinetics or therapies. For example, these profiles may be created and evaluated for labor and blood samples prior to the patient's being treated or during treatment to determine if there are nucleic acids or genes whose expression correlates with the outcome of the patient's treatment. Identification of differential nucleic acids or genes can lead to a diagnostic assay for evaluation of samples to determine what drug regimen the patient should be provided. In addition, it can be used to identify or select patients suitable for a particular clinical trial. If an expression profile is determined to be correlated with drug efficacy or drug toxicity that profile is relevant to whether that patient is an appropriate patient for receiving a drug, for receiving a combination of drugs, or for a particular dosage of the drug.

C. Other Assays

In addition to the use of arrays and microarrays, it is contemplated that a number of different assays could be employed to analyze nucleic acids or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### VI. Methods for Evaluating a Subject

The current invention provides methods for the detection, diagnosis, monitoring, and prognosis of labor, preterm labor, onset of preterm labor, or status of preterm labor involve the measurement of one or more biomarkers. The methods may also be used to identify subjects with a predisposition to preterm labor. In accordance with a method of the invention, the quantitated levels of one or more biomarkers in a patient are compared to levels of the comparable selection of biomarkers quantitated for one or more control samples (e.g. normal, without preterm labor) wherein a change in the levels of one or more biomarkers levels is the basis for identifying the patient as being at risk for beginning preterm labor. In certain aspects, a method is provided for identifying at-risk preterm patients using one or more biomarkers, which method can be shown by regression analysis to have useful negative and positive predictive power, particularly adequate sensitivity to changes in selected biomarker levels, adequate specificity of biomarker changes to individual stages of preterm labor, and a strong enough correlation between biomarker changes and the initiation of preterm labor.

In an aspect of the invention, a method for detecting preterm labor is provided comprising: (a) obtaining a sample suspected of containing one or more biomarkers; (b) contacting the sample with antibodies that specifically bind to the biomarkers under conditions effective to form complexes; (c) measuring the amount of biomarkers present in the sample by quantitating the presence and/or amount of the complexes; and (d) comparing the amount of biomarkers present in the samples with the amount of biomarkers in a control or to a reference or standard. Typically, a change or significant difference in the amount of biomarkers in the sample compared with the amount in the control is indicative of a risk of preterm labor or the initiation of preterm labor.

In an embodiment, the invention contemplates a method for monitoring the progression of preterm labor in an individual, comprising: (a) contacting antibodies which bind to one or more biomarkers with a sample from the individual so as to form complexes comprising the antibodies and one or more biomarkers in the sample; (b) determining or detecting the presence or amount of complex formation in the sample; (c) repeating steps (a) and (b) at a point later in time; and (d) comparing the result of step (b) with the result of step (c), wherein a difference in the amount of complex formation is indicative of ongoing preterm labor. The amount of complexes may also be compared to a value representative of the amount of the complexes from an individual not at risk of, or afflicted with preterm labor. A significant difference in complex formation may be indicative of active or progressing preterm labor in the individual, and potentially, imminent preterm birth.

In further aspects, a method for identifying an individual at risk for preterm labor is provided comprising: (a) obtaining a sample from an individual, typically the individual will be suspected of being at risk for preterm labor; (b) contacting the sample with antibodies or binding agents that specifically bind to the biomarkers under conditions effective to form biomarker complexes; (c) measuring the amount of biomarkers present in the sample by quantitating the amount of the complexes; and (d) comparing the amount of biomarkers present in the samples with the amount of biomarkers in a control or with a reference or standard, wherein a change or significant difference in the amount of biomarkers in the sample compared with the amount in the control or the reference is indicative of an increased risk or probability of preterm labor.

Differential levels of biomarkers measured in a patient sample can indicate an increased risk of preterm delivery, particularly before 37 weeks gestation, more particularly before 34 weeks gestation. In other embodiments, an increased level of one or more biomarkers measured in a patient sample can indicate an increased risk of preterm delivery in less than 48 hours from clinical presentation. In some of these embodiments, a preterm delivery is predicted within 48 hours when disparities are measure in the levels of one or more biomarkers selected from serum retinol binding protein (RBP4), apolipoprotein C1 (ApoC1), pre-B-cell colony-enhancing factor (PBEF), interleukin 6 (IL-6), interleukin 8 (IL-8), relaxin, caspase recruitment domain containing protein CARD 12, and/or ferritin, including all combinations and permutations thereof. In certain embodiments, the PTL biomarkers useful in predicting imminent (i.e. less than 48 hours in the future) labor in a preterm patient are RBP4, ApoC1, PBEF, IL-8, and ferritin.

The invention contemplates a method for detecting or monitoring the stage or type of preterm labor or onset of preterm labor, comprising producing a profile of levels of one or more PTL biomarker and optionally other biomarkers associated with pretern labor or labor in a sample from a patient, and comparing the profile with a reference to identify a profile for the patient indicative of the stage or type of labor or preterm labor.

The methods described herein may be used to evaluate the probability of the presence of labor, preterm labor, or premature onset of labor, for example, in a sample freshly removed from a subject. Such methods can be used to detect labor and/or preterm labor and help in the diagnosis and prognosis of labor and other pregnancy complications. The methods can be used to detect the potential for preterm labor and to monitor preterm labor or a therapy.

The invention also contemplates a method for detecting preterm labor or onset of preterm labor comprising producing a profile of levels of one or more biomarker and other biomarkers associated with preterm labor in a sample (e.g. blood) from a patient, and comparing the profile with a reference to identify a profile for the patient indicative of preterm labor.

The methods described herein can be adapted for diagnosing and monitoring preterm labor by detecting one or more biomarkers in biological samples from a subject. These applications may use the amount of biomarkers quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of labor or preterm labor, may be established by prospective and/or retrospective statistical studies. Healthy subjects who have no clinically evident preterm labor or abnormalities may be selected for statistical studies. Diagnosis may be made by a finding of statistically different levels of detected biomarkers associated with preterm labor, compared to a control sample or previous levels quantitated for the same subject.

The methods described herein may also use multiple biomarkers for labor and/or preterm labor. Therefore, the invention contemplates a method for analyzing a biological sample for the presence of one or more biomarkers, and other biomarkers that are specific indicators of labor and/or preterm labor. The methods described herein may be modified by including reagents to detect the additional biomarkers.

### VII. Kits, Devices, and Implements

The invention also contemplates kits, devices, and implements for carrying out the methods of the invention. Kits may typically comprise two or more components required for performing a diagnostic assay. Components include but are not limited to compounds, reagents, containers, and equipment.

The methods described herein may be performed by utilizing pre-packaged diagnostic kits comprising one or more biomarker or biomarker binding agent (e.g., antibody) described herein, which may be conveniently used in clinical settings for example. Typically, the kits of the invention can be used to screen and diagnose patients for the presence or absence of markers for labor and/or preterm labor. Also, subjects may be screened or assessed to identify those individuals having a predisposition to developing preterm labor.

A container, device or implement within a kit can comprise a binding agent or reagent as described herein. By way of example, the kit may contain antibodies or antibody fragments which bind specifically to one or more biomarkers, and optionally other biomarkers. A kit may also include antibodies against the biomarker antibodies labeled with an enzyme, and a substrate for the enzyme. The kit may also contain chromatographic substrate, peptide arrays, antibody arrays, microtiter plate(s), standards, assay diluent, wash buffer, adhesive plate covers, and/or instructions for carrying out a method of the invention using the kit.

In an aspect of the invention, the kit includes antibodies or fragments of antibodies that bind specifically to one or more biomarkers and means or labeling scheme for detecting the binding of the antibodies to their respective biomarkers. Antibodies or detection reagents can be supplied as concentrates (including lyophilized compositions) that can be further diluted prior to use or at a concentration for use.

A kit may be designed for detection or measurement of biomarkers in a patient sample. In certain aspects the biomarkers include

In a further aspect, a kit contains a protein chip or protein array or antibody array coupled with biomarker antibodies or binding agents. Such components can be configured and ready to be contacted with a patient sample or a normal sample or standard. Also, software for the data analysis can be included. The software can comprise data analysis methods, in particular mathematical routines for biomarker detection, evaluation, measurement, and/or discovery, including the calculation of correlation coefficients between clinical categories and biomarker measurements. The software may also include mathematical routines for calculating the correlation between sample biomarker concentration and control biomarker concentration, using array-generated fluorescence data, to determine the clinical classification of the sample.

The invention contemplates a kit for assessing the presence or absence of biomarker indicative of labor and/or preterm labor and/or onset of preterm labor. The kit can comprise antibodies specific for one or more biomarkers, and optionally probes, primers or antibodies specific for other biomarkers associated with labor and/or preterm labor or other conditions related to pregnancy.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of Biomarkers

Currently, there are two tests used to determine if preterm labor will result in a preterm birth: however, both fetal fibronectin and cervical length are best suited as negative predictors. A novel diagnostic platform has recently been described in the literature using two specific technologies: high-throughput SELDI (Surface-Enhanced Laser Desorption Ionization) ProteinChip (Ciphergen) coupled with a time-of-flight mass spectrometer. The data generated by these technologies contains many thousands of data points and requires various bioinformatic algorithms capable of discriminating a disease state from normal. The study was to assess the applicability of SELDI, Time-of-flight mass spectroscopy, and a readily commercially available Bioinformatics tool to analyze the low mass portion of the maternal serum proteome to distinguish labor from non-labor in the term and preterm period.

Serum and clinical data collection. Serum was collected from women presenting at Kapiolani Medical Center for Women and Children while in labor or being ruled out for labor (non-labor) during both the term and preterm period (<36 weeks). Serum was processed within 10 minutes of collection and aliquoted into three to four screw-top microfuge tubes. Clinical data was collected retrospectively via chart review by a perinatologist and collated by a research nurse. 15 specific clinical data points were collected. These included patients age, race, gestational age, Time of presentation, Time of serum collection, concomitant medical conditions, medications, length of labor and pregnancy outcome among others. For the study, 50 patients in the labor and 50 non-labor controls were utilized.

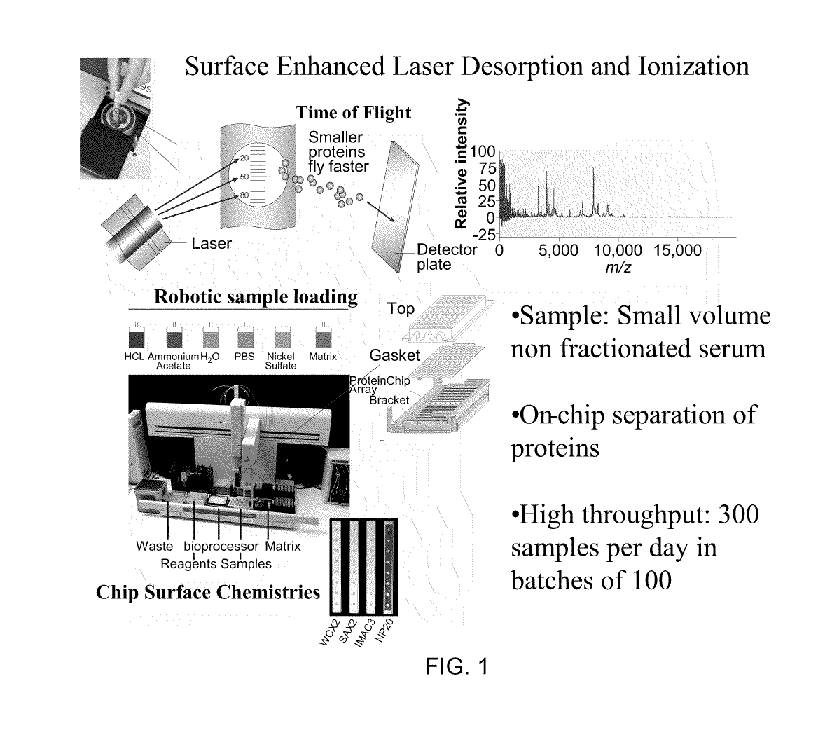

Proteomic Analysis. Serum samples were thawed, added to a IMAC interaction protein chip, and analyzed on the Protein Biology System 2 SELDI-TOF mass spectrometer (FIG. 1) (Ciphergen Biosystems, Freemont, Calif., USA). Mass resolution (defined as m/Δm) is routinely achieved below 400. Mass accuracy is assessed daily through the use of angiotensin peptide calibrations. The inventors achieved a mass accuracy of 0·1% with this system. Peptides and proteins below the 20,000 mass/charge (M/Z) range were ionized with -cyano-4-hydroxycinnamicacid as a matrix, which is most effective for the detection of proteins and peptides in this mass range. The chips were analyzed manually under the following settings: laser intensity 240, detector sensitivity 10, mass focus 6000, position 50, molecular mass range 0-20000 Da, and a 50-shot average per sample. Data were collected without filters and were later used for analyses. Labor and non-labor samples and controls were run concurrently, intermingled on the same chip; the operators were blinded to sample identity.

Data Analysis. Data analysis was performed using the GeneSping analysis package by SiliconGenetics, with data handling, filtering, and analysis methods developed in house. Spectra were imported into the software in microarray-style format. M/Z values were designated as gene names and data point amplitudes as signal intensities with the metadata entered as parameters. Our first goal was to eliminate peaks with uninteresting patterns. Several filtering tools were utilized, including K-means clustering, and uninformative peaks were removed from subsequent analysis. Hierarchical clustering was used to determine the relationship of samples to each other to in sure that there were two predicted classes (labor and non-labor). In identifying a set of predictor peaks, the inventors applied Fisher's exact test, and identified 25 peaks potentially useful for discriminating labor from non-labor. Training data was also cross-validated using a K-nearest neighbor algorithm with five nearest neighbor's used for voting. A parameter predictor algorithm was used to test the model. The model was iteratively refined using a metadata-based analysis to minimize the bias in the predictors caused by intra-group and population heterogeneity before arriving at the optimum predictor set for each comparison.

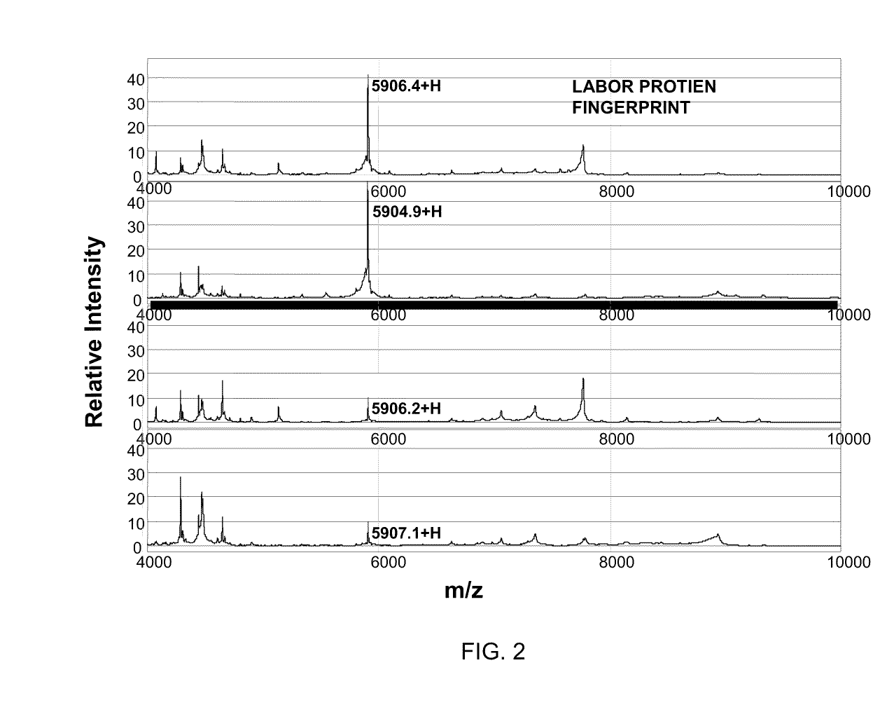

Results. In the full dataset, with a simple analysis, the inventors were able to discriminate labor from false labor in the term and pre-term period with 82% sensitivity and 76% specificity using 25 data points. One particular peak at 5908.6 m/z was able to model with an accuracy of 60% with 12 false positives and 10 false negatives (Table 1). Based on this single value, it was clear that there was poor separation of the labor and non-labor groups when the average peak amplitude of 5908.6 m/z plus are compared using the entire population. In FIG. 2, the bold line represents a plus one standard deviation from the mean.

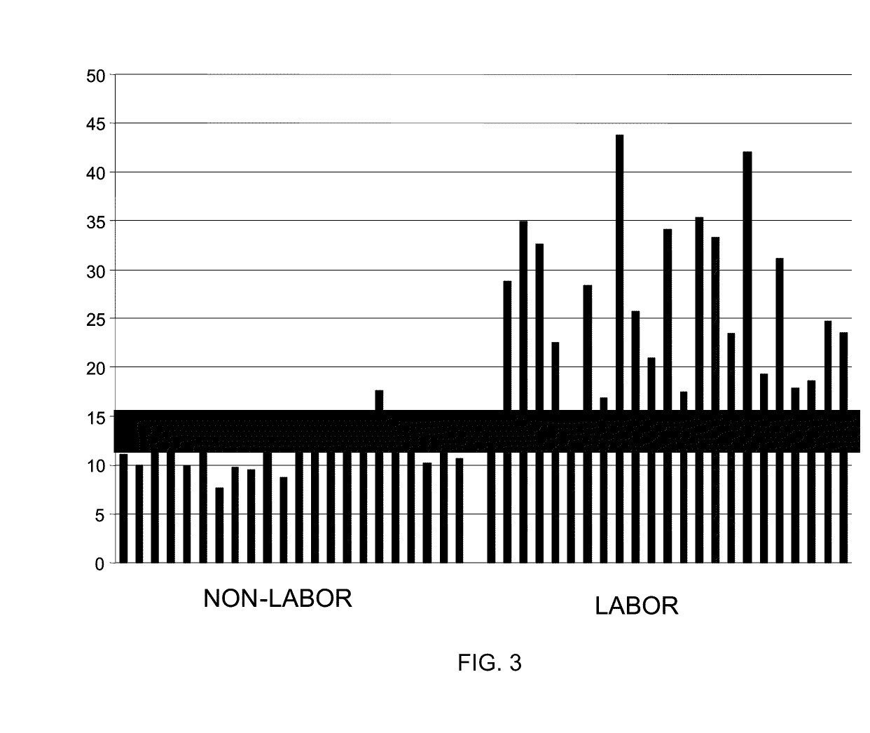

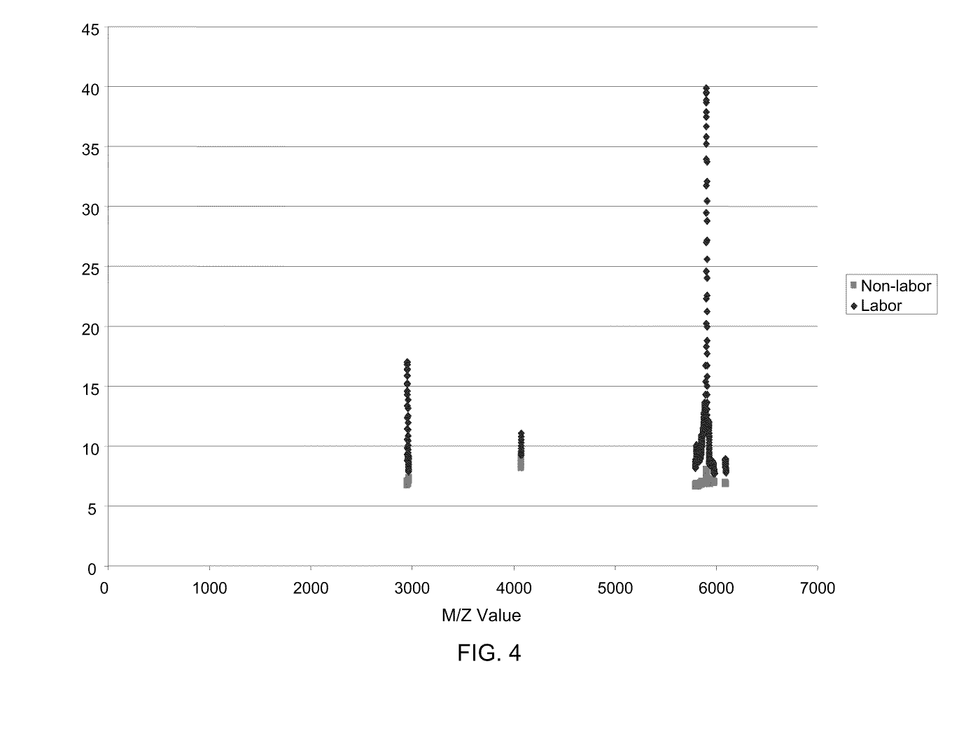

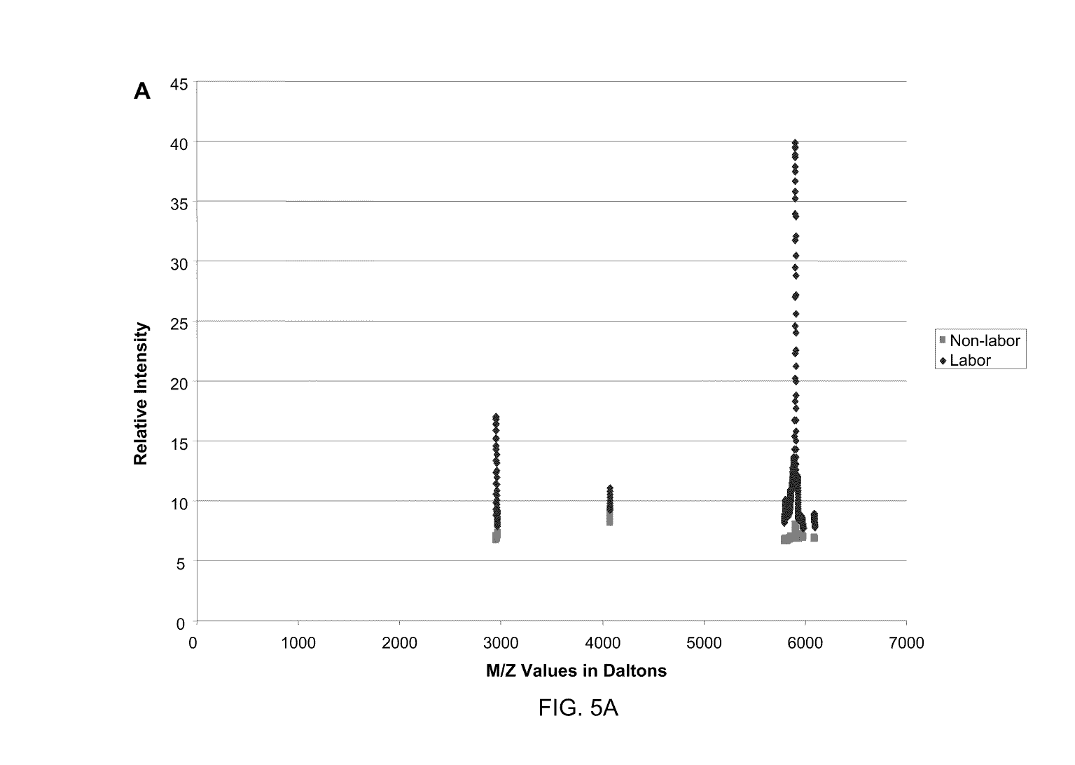

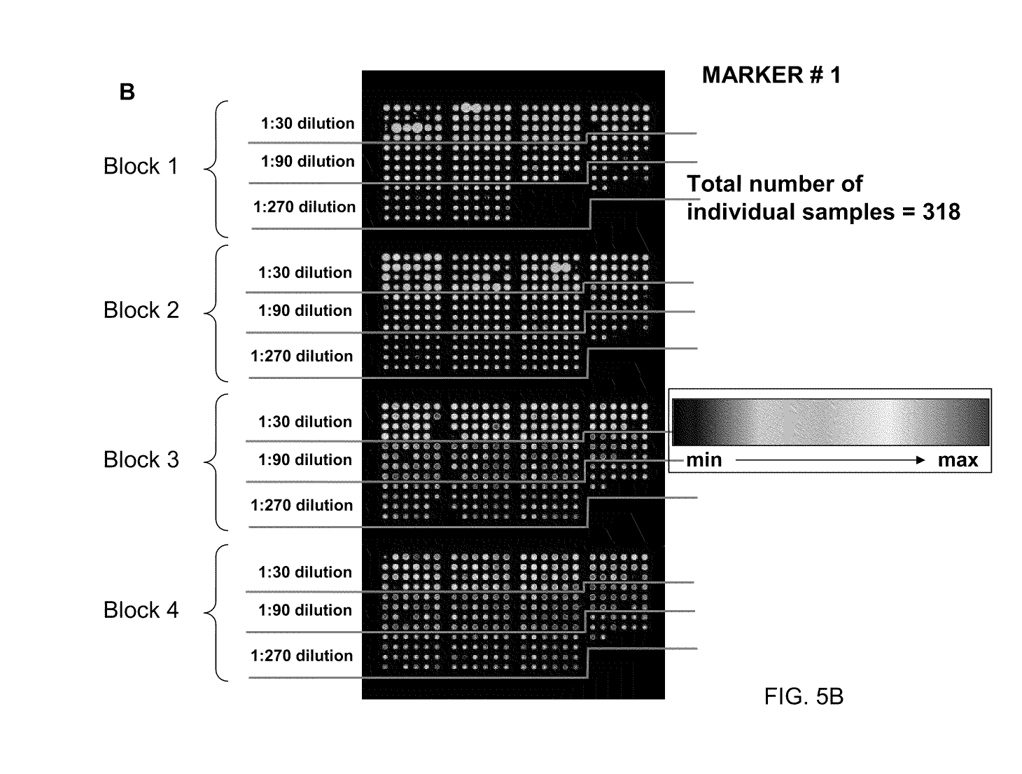

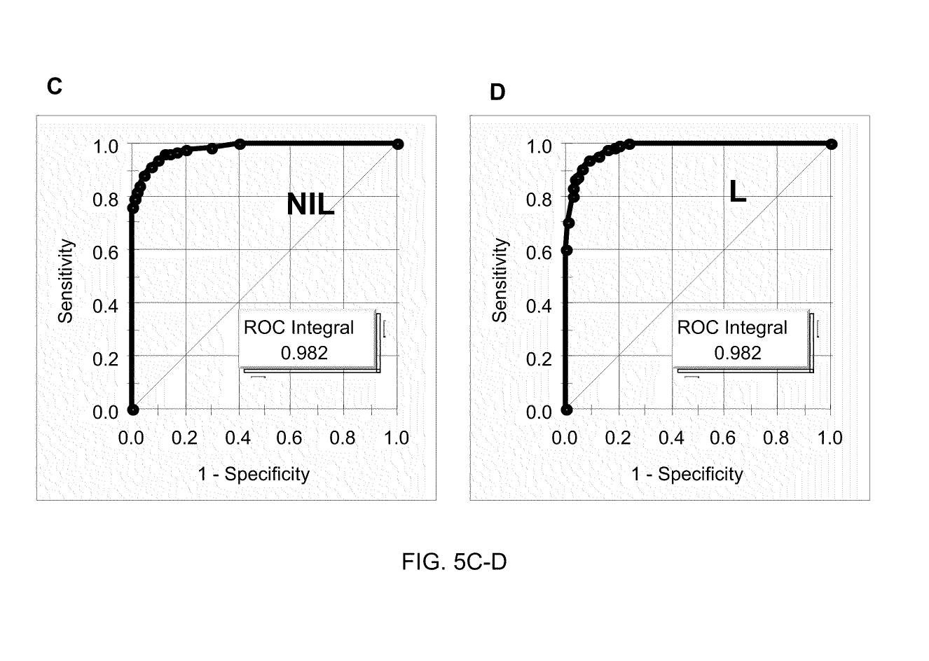

To improve this model, two specific ethnic groups were excluded from analysis. Caucasians and African-Americans, representing less than 10% of the study population were excluded. Focusing on the Asian and Pacific Islander group, while not improving overall diagnostic accuracy of the 5908.6 peak, average peak intensity of the labor group rose to 28.1 from a previous level of 23.3. Additionally, the clinical data of the 12 false positive and 10 false negative patients were examined. Ten of 12 patients predicted to be in labor, and not delivering within 24 hours all had clinical signs of infection (maternal fever). Eight of 10 patients that were in labor and delivering within 24-hour that were not predicted to be in labor had vaginal bleeding. Of the two remaining false positive patients, one had pancreatitis and one had preeclampsia. When corrected for maternal infection, the mean amplitude of 5908.6 value for the non-labor group dropped from 16.1 to 12.4. The algorithm was re-trained to assess for labor (delivery within 24 hours) or intrauterine infection in the Asian/Pacific Islander population. The result (FIG. 3) increased the predicted accuracy of the 5908.6 m/z value to 96%. Lastly, two other m/z values from the original 25 data points were used in the final model (2953.4 and 4125.6) (FIG. 4). This further improved diagnostic accuracy to 98% (Table 2).

TABLE 1
Biological parameters

| Non-labor parameter | Amplitude | Number of patients |
|---|---|---|
| infection | 24.8 | 12 |
| Delivery <24 hrs | 23.3 | 7 |
| Bleeding | 14 | 10 |

TABLE 2
Comparison of Preterm Labor Tests

| Test Name | NPV | PPV | TPV |
|---|---|---|---|
| Fetal Fibronectin* | 99% | 16.70% | 56% |
| Proteomic Test | 98% | 96% | 97% |

*Values from Bernhardt J and Dorman K

In preliminary work on over 300 patients, half of whom imminently went into labor, the inventors have determined the identities of several novel markers that will provide actual clinical utility. First of all, the markers are all measured as serum (a specially prepared fraction of collected blood) tests thereby making it much easier and less costly for clinicians and patients to have testing performed-i.e. gynecologic exams to perform cervical swabs are unnecessary. The first marker (designated PTB#1 in Table 3; the other markers are numbered accordingly) by itself does as well, even in its initial configuration, as a single marker test as that achieved by fFN after more than a decade of optimization with its use.

For a portfolio of markers, identified by proteomic analysis, the inventors have achieved much higher sensitivities (the proportion of persons with the condition who test positive) than fFN for the same false positive rates. The data show far higher sensitivities and PPVs when the specificity was held constant across other markers including fFN-this comparison therefore shows that our test actually may identify a population at high risk for PTB with a sensitivity of 88%.

TABLE 3
The PTB marker multivariate and univariate Analyses. The
table lists the sensitivity (Sens), specificity (Spec), positive
predictive value (PPV) and negative predictive value (NPV)
for five of the markers individually and when used as a panel
(only the five markers contributing to the panel are shown).
fFN is listed by comparison. The Spec for each marker was
set near that for fFN and the Sens, PPV, and NPV were
determined to compare to fFN.

| | SENS | SPEC | PPV | NPV |
|---|---|---|---|---|
| Multi-panel | 88% | 95% | 72% | 98% |
| PTB#1 | 39% | 96% | 37% | 98% |
| PTB#2 | 77% | 94% | 64% | 97% |
| PTB#3 | 80% | 95% | 67% | 97% |
| PTB#5 | 54% | 95% | 58% | 94% |
| PTB#8 | 65% | 95% | 30% | 92% |
| fFN (IOM) | 39% | 95% | 30% | 92% |

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,282,287
U.S. Pat. No. 4,542,102
U.S. Pat. No. 4,582,788
U.S. Pat. No. 4,656,127
U.S. Pat. No. 4,659,774
U.S. Pat. No. 4,682,195
U.S. Pat. No. 4,683,194
U.S. Pat. No. 4,683,195
U.S. Pat. No. 4,683,202
U.S. Pat. No. 4,800,159
U.S. Pat. No. 4,816,571
U.S. Pat. No. 4,879,236
U.S. Pat. No. 4,883,750
U.S. Pat. No. 4,946,773
U.S. Pat. No. 4,959,463
U.S. Pat. No. 5,141,813
U.S. Pat. No. 5,143,854
U.S. Pat. No. 5,202,231
U.S. Pat. No. 5,242,974
U.S. Pat. No. 5,252,743
U.S. Pat. No. 5,264,566
U.S. Pat. No. 5,279,721
U.S. Pat. No. 5,288,644
U.S. Pat. No. 5,324,633
U.S. Pat. No. 5,354,855
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,582,981
U.S. Pat. No. 5,593,839
U.S. Pat. No. 5,599,672
U.S. Pat. No. 5,599,695
U.S. Pat. No. 5,602,244
U.S. Pat. No. 5,610,287
U.S. Pat. No. 5,624,711
U.S. Pat. No. 5,631,134
U.S. Pat. No. 5,639,603
U.S. Pat. No. 5,645,897
U.S. Pat. No. 5,650,298
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,705,629
U.S. Pat. No. 5,744,305
U.S. Pat. No. 5,756,291
U.S. Pat. No. 5,780,610
U.S. Pat. No. 5,792,613
U.S. Pat. No. 5,800,992
U.S. Pat. No. 5,807,522
U.S. Pat. No. 5,830,645
U.S. Pat. No. 5,837,196
U.S. Pat. No. 5,840,867
U.S. Pat. No. 5,843,650
U.S. Pat. No. 5,846,709
U.S. Pat. No. 5,846,783
U.S. Pat. No. 5,847,219
U.S. Pat. No. 5,849,497
U.S. Pat. No. 5,849,546
U.S. Pat. No. 5,849,547
U.S. Pat. No. 5,858,652
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,912,148
U.S. Pat. No. 5,916,776
U.S. Pat. No. 5,916,779
U.S. Pat. No. 5,919,626
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,368,799
U.S. Pat. No. 6,383,749
U.S. Pat. No. 6,617,112
U.S. Pat. No. 6,638,717
U.S. Pat. No. 6,720,138
ACOG. American College of Obstetricians and Gynecologists Practice Bulletin. Dystocia and augmentation of labor. Clinical management guidelines for obstetricians-gynecologists. No 49. Washington, D.C. American College of Obstetricians and Gynecologists;. December 2003.
Albers et al., Obstet Gynecol. March 1996; 87(3):355-9.
Cheng et al., Am J Obstet Gynecol. September 2004; 191(3):933-8.
Friedman Obstet Gynecol. December 1955; 6(6):567-89.
Friedman and Sachtleben, Obstet Gynecol. February 1961a; 17:135-48.
Friedman and Sachtleben, Obstet Gynecol. May 1961b; 17:566-78.
Janni et al., Acta Obstet Gynecol Scand. March 2002; 81(3):214-21.
Kilpatrick and Laros, Obstet Gynecol. July 1989; 74(1):85-7.
Menticoglou et al., Am J Obstet Gynecol. September 1995; 173(3 Pt 1):906-12.
Myles and Santolaya, Obstet Gynecol. July 2003; 102(1):52-8.
Norwitz et al., Labor and delivery. In: Gabbe S G, Niebyl J R, Simpson J L, eds. Obstetrics: Normal and problem pregnancies. 3rd ed. New York. Churchill Livingstone;. 2003.
O'Connell et al., J Obstet Gynaecol. May 2003; 23(3):255-7.
Senecal et al., Obstet Gynecol. April 2005; 105(4):763-72.
Zhang et al., Am J Obstet Gynecol. October 2002; 187(4):824-8.
Abdel-Rahman et al., J Biochem. Toxicol., 9:191-198, 1994.
Al-Hendy and Luxon, J. Soc. Gynecol. Investig., 9(1):744, 2002.
Al-Hendy et al., J. Soc. Gynecol. Investig., 9(1):794, 2002.
Andersen et al., J. Soc. Gynecol. Investig., 2:542-551, 1995.
Axelrod and Tomchick, J. Biol. Chem., 233:702-705, 1958.
Baichwal and Sugden, In: Gene Transfer, Kucherlapati (Ed.), NY, Plenum Press, 117-148, 1986.
Ball and Knuppen, Acta Endocrinol. Suppl. (Copenh), 232:1-127, 1980.
Balloch, In: The relative frequency of fibroid processes in the dark-skinned races, Medical News, 29-35, 1984.
Banerjee et al., Neoplasia, 5(5):417-426, 2003.
Banerjeei et al., Anticancer Res., 20(4):2641-2645, 2000.
Barany and Merrifield, In: The Peptides, Gross and Meienhofer (Eds.), Academic Press, NY, 1-284, 1979.
Barchiesi et al., Hypertension., 42:420, 2003.
Bellus, J. Macromol. Sci. Pure Appl. Chem., A31(1): 1355-1376, 1994.
Berzal-Herranz et al., Genes Dev, 6(1):129-134, 1992.
Brandon et al., J. Clin. Endocrinol. Metab., 80:1876-1881, 1995.
Brummelkamp et al., Science, 296(5567):550-553, 2002.
Bruno et al., Lancet. Oncol., 5(7):430-442, 2004.
Burgstaller et al., Curr. Opin. Drug Discov. Devel., 5(5):690-700, 2002.
Buttram and Reiter, Fertil. Steril., 36:433-445, 1981.
Carlson et al., Obstet. Gynecol., 83:556-565, 1994.
Carlson et al., Obstet. Gynecol., 83:566-572, 1994.
Cech et al., Cell, 27(3 Pt 2):487-496, 1981.
Chegini et al., J. Soc. Gynecol. Investig., 10:161-171, 2003.
Chiaffarino et al., Br. J. Obstet. Gynaecol., 106:857-860, 1999.
Chiaffarino et al., Obstet. Gynecol., 94:395-398, 1999.
Chowrira et al., J. Biol. Chem., 268:19458-62, 1993.
Chowrira et al., J. Biol. Chem., 269(41):25856-25864, 1994.
Coligan et al., Current Protocols in Immunology, 1(2): Chapter 5, 1991.
Cook and Walker, Semin. Reprod. Med., 22(2):105-111, 2004.
Coronado et al., Obstet. Gynecol., 95:764-769, 2000.
Coupar et al., Gene, 68:1-10, 1988.
Cramer and Patel, Am. J. Clin. Pathol., 94:435-438, 1990.
Culver et al., Science, 256(5063):1550-1552, 1992.
Dawood et al., Fertil. Steril., 52:21-26, 1989.
Deligdish and Loewenthal, J. Clin. Pathol., 23:676-680, 1970.
Dubey et al., Arterioscler. Thromb. Vasc. Biol., 20:964-972, 2000.
Dunlap, In: Immobilized Biochemicals and Affinity Chromatography, Adv. Exp. Med. Biol. 42, Plenum Press, N.F. 1974
Elbashir et al., Genes Dev., 5(2):188-200, 2001.
European Appln. 201,184
European Appln. 237,362
European Appln. 258,017
European Appln. 266,032
European Appln. 320 308
European Appln. 329 822
European Appln. 373 203
European Appln. 50,424
European Appln. 785 280
European Appln. 799 897
European Appln. 84,796
Feigelson and Henderson, Carcinogenesis, 17(11):2279-2284, 1996.
Figg et al., Invest. New Drugs, 20(2):183-194, 2002.
Flake et al., Environ. Health Perspect., 11 1(8):1037-1054, 2003.
Forster and Symons, Cell, 49(2):211-220, 1987.
French Appln. 2,650,840
Friedmann, Science, 244:1275-1281, 1989.
Frisch et al., Mol. Psychiatry, 6:243-245, 2001.
Froehler et al., Nucleic Acids Res., 14(13):5399-5407, 1986.
Frohman, In: PCR Protocols: A Guide To Methods And Applications, Academic Press, N.Y., 1994.
Fuchs-Young et al., Mol. Carcinog., 17:151-159, 1996.
GB Appln. 2 202 328
GB Appln. 8 803 000
Gerlach et al., Nature (London), 328:802-805, 1987.
Ghosh et al., Melanoma Res., 13(2):119-127, 2003.
Goodman et al., Carcinogenesis, 22:1661 -1665, 2001.
Gordon etal., Curr. Opin. Obstet. Gynecol., 15(5):377-384., 2003.
Gossen and Bujard, Proc. Natl. Acad. Sci. USA, 89:5547-5551, 1992.
Gossen et al., Science, 268(5218):1766-1769, 1995.
Gupta et al., J. Steroid Biochem. Mol. Biol., 67(5-6):413-419, 1998.
Harlow and Lane, In: Antibodies. A Laboratory Manual, Cold Spring Harbor Laboratory, Cold Spring Harbor, N.Y., 346-348, 1988.
Haseloff and Gerlach, Nature, 334:585-591, 1988.
Hidalgo et al., Adv. Intern Med., 47:159-190, 2001.
Horwich et al. J. Virol., 64:642-650, 1990.
Houghten et al., Infect. Immun., 48(3):735-740., 1985.
Howe et al., Am. J. Pathol., 146:1568-1579, 1995.
Howe et al., Endocrinology, 136:4996-5003, 1995.
Hsieh et al., Fertil. Steril., 79:96-99, 2003.
Huang et al., Cancer Res., 59:4870-4875, 1999.
Hunter et al., Environ. Health Perspect., 108(5):829-834, 2000.
Jakoby and Wilchek, Methods Enzymol., 34:3-10, 1974.
Joyce, Nature, 338:217-244, 1989.
Kim and Cech, Proc. Natl. Acad. Sci. USA, 84(24):8788-92, 1987.
Kitawaki et al., Hum. Reprod., 16:51-55, 2001.
Kjerulff et al., J. Reprod. Med., 41:483-490, 1996.
Kjerulffet al., Obstet. Gynecol., 82:757-764, 1993.
Kjerulffet al., Obstet. Gynecol., 95:319-326,2000.
Komher, et al., Nucl. Acids. Res. 17:7779-7784, 1989.
Kuppuswamy, et al., Proc. Natl. Acad. Sci. USA, 88:1143-1147,1991.
Lachman et al., Pharmacogenetics, 6:243-250, 1996.
Landegren, et al., Science 241:1077-1080, 1988.
Lavigne et al., Cancer Res., 57:5493-5497, 1997.
Lethaby et al., Update Software Ltd., The Cochrane Library, 2001.
Lieber and Strauss, Mol. Cell Biol., 15(1):540-551, 1995.
Linde, In: Operative Gynecology, Rock and Thompson (Eds.), Philadelphia: Lippincott-Raven; 732, 1997.
Mabjeesh et al., Cancer Cell, 3(4):363-375, 2003.
Marshall et al., Epidemiology, 9:511-517, 1998.
Marshall et al., Fertil. Steril., 70:432-439, 1998.
Marshall et al., Obstet. Gynecol., 90:967-973, 1997.
Martel et al., Semin. Reprod. Med., 22(2):91-103, 2004.
Massart et al., Fertil. Steril., 75:567-570, 2001.
Matsuo et al., J Clin Endocrinol Metab. 82:293-299, 1997.
Maxam, et al., Proc. Natl. Acad. Sci. USA, 74:560, 1977.
McLachlan et al., Br. J. Obstet. Gynecol., 93:431-454, 1986.
McLeod et al., J. Pharmacol. Exp. Ther., 270:26-29, 1994.
Merrifield, Science, 232(4748):341-347, 1986.
Michel and Westhof, J. Mol. Biol., 216:585-610, 1990.
Mitrunen et al., Cancer Epidemiol. Biomarker Prev., 10:635-640, 2001.
Mooberry, Curr. Opin. Oncol., 15(6):425-430, 2003.
Mooberry, Drug Resist Updat., 6(6):355-361, 2003.
Mullis et al., Cold Spring Harbor Symp. Quant. Biol. 51:263-273, 1986.
Nickerson et al., Proc. Natl. Acad. Sci. USA, 87:8923-8927,1990.
Nicolas and Rubenstein, In: Vectors. A survey of molecular cloning vectors and their uses, Rodriguez and Denhardt (Eds.), Stoneham: Butterworth, 494-513, 1988.
Nowak, Clin. Obstet. Gynecol., 44(2):327-334, 2001.
Nowak, Environ. Health Perspect., 108(5):849-853, 2000.
Nyren et al., Anal. Biochem. 208:171-175, 1993.
Ohara et al., Proc. Natl. Acad. Sci. USA, 86:5673-5677, 1989.
Palukaitis et al., Virology, 99:145-151, 1979.
Parazzini et al., Epidemiology, 7:440-442, 1996.
Parazzini etal., J. Reprod. Med., 41:316-320, 1996.
Patrikis et al., Mol. Carcinog., 37:61-64, 2003.
PCT Appln. PCT/US87/00880
PCT Appln. PCT/US89/01025
PCT Appln. WO 0138580
PCT Appln. WO 0168255
PCT Appln. WO 03/012052
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 84/03564
PCT Appln. WO 88/10315
PCT Appln. WO 89/06700
PCT Appln. WO 90/07641
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 99/23256
PCT Appln. WO 99/35505
PCT Appln. WO 99/36760
PCT Appln. WO91/02087
PCT Appln. WO92/15712
PCT Pubn. 90/07582
PCT Pubn. 91/00868
PCT Pubn. 91/07087
Perriman et al., Gene, 113:157-163, 1992.
Perrotta and Been, Biochemistry, 31(1):16-21, 1992.
Pollow et al., J. Clin. Chem. Clin. Biochem., 16:493-502, 1978.
Prezant et al., Hum. Mutat., 1:159-164, 1992.
Prody et al., Science, 231:1577-1580, 1986.
Reddy et al., Steroids, 37:195-203, 1981.
Reenila, In: Catechol-O-Methyltransferase Activity: Assay, Distribution and Pharmacological Modification, Helsinki, 1999.
Reinhold-Hurek and Shub, Nature, 357:173-176, 1992.
Remington's Pharmaceutical Sciences, 18th Ed. Mack Printing Company, pp. 1289-1329, 1990.
Rice et al., Am. J. Obstet. Gynecol., 160:1212-1216, 1989.
Ridgeway, In: Vectors: A survey of molecular cloning vectors and their uses, Rodriguez and Denhardt (Eds.), Stoneham:Butterworth, 467-492, 1988.
Ross et al., BMJ, 293:359-362, 1986.
Rowe et al., Obstet. Gynecol., 93:915-921, 1999.
Sambrook et al., In: Molecular cloning, Cold Spring Harbor Laboratory Press, Cold Spring Harbor, N.Y., 2001.
Sanger, et al., J. Molec. Biol., 94:441, 1975.
Sarver et al., Science, 247:1222-1225, 1990.
Scanlon et al., Proc. Natl. Acad. Sci. USA, 88:10591-10595, 1991.
Schneider et al., J. Biol. Chem., 259(8):4840-4845, 1984.
Schutze et al., J. Steroid Biochem. Mol. Biol., 46(6):781-789, 1993.
Schwartz et al., Environ. Health Perspect., 108(5):821-827, 2000.
Sioud et al., J. Mol. Biol., 223:831-835, 1992.
Sokolov, Nucl. Acids Res. 18:3671, 1990.
Stewart and Young, In: Solid Phase Peptide Synthesis, 2d. ed., Pierce Chemical Co., 1984.
Stewart et al., J. Clin. Endocrinol. Metab., 79:900-906, 1994.
Sui et al., Proc. Natl. Acad. Sci. USA, 99(8):5515-5520, 2002.
Swaneck et al., Proc. Natl. Acad. Sci. USA, 85(21):7831-7835, 1988.
Symons, Annu. Rev. Biochem., 61:641-671, 1992.
Symons, Nucl. Acids Res., 9(23):6527-6537, 1981.
Syvanen et al., Am. J. Hum. Genet., 52(1):46-59, 1993.
Syvanen et al., Genomics, 8:684-692, 1990.
Tam et al., J. Am. Chem. Soc., 105:6442, 1983.
Temin, In: Gene Transfer, Kucherlapati (Ed.), NY, Plenum Press, 149-188, 1986.
Thompson et al., Cancer Res., 58, 2107-2110, 1998.
Thompson et al., Nature Genet., 9:444-450, 1995.
Torpin et al., Am. J. Obstet. Gynecol., 44:569-574, 1942.
Tsibris et al., Fertil. Steril., 78:114-121, 2002.
Ugozzoll et al., GATA 9:107-112, 1992.
Van Aswegen et al., J. Steroid Biochem., 32(4):485-492, 1989.
Vandewalle et al., Mol. Cell Endocrinol., 61(2):239-246, 1989.
Walker et al., Genes Chromosomes Cancer, 38(4):349-356, 2003.
Walker et al., Genes Chromosomes Cancer, 38(4):349-356, 2003.
Walker et al., Proc. Natl. Acad. Sci. USA, 89:392-396 1992.
Walker, Recent Prog. Horm. Res., 57:277-294, 2002.
Weismiller, Am. Fam. Physician, 59(3):593-602, 1999.
Wilcox et al., Obstet. Gynecol., 83:549-555, 1990.
Wilson, In: Polycystic Kidney Disease, Watson and Torres (Eds.), Oxford Medical Publications; 125-163, 1996.
Witherspoon and Butler, Surg. Gynecol. Obstet., 58:57-61, 1934.
Xie et al., Proc. Am. Assoc. Cancer Res., 40:3744, 1999.
Yim et al., Pharmacogenetics, 11:279-286, 2001.
Yuan and Altman, Science, 263:1269-1273, 1994.
Yuan et al., Proc. Natl. Acad. Sci. USA, 89:8006-8010, 1992.
Zacharia et al., Circulation, 108(24):2974-2978, 2003.
Zacharia et al., J. Pharmacol. Exp. Ther., 309(3):1093-1097, 2004.
Zaloudek and Hendrickson In: Blaustein's. Pathology of the Female Fenital Tract, Springer-Verlag (Ed.), 5^{th} Ed., NY, 561-615, 2002.
Zhu and Connery, Cancer Res., 58:2269-2277, 1998.
Zhu and Conney, Carcinogenesis, 19:1-27, 1998.
Zhu and Conney, Carcinogenesis, 19:1-27, 1998.
Zhu, Curr Drug Metab., 3(3):321-349, 2002.

1. A method of detecting preterm labor comprising detecting a biomarker complex of serum retinol binding protein (RBP4) and/or apoliprotein C1 (ApoC1) in a sample from a pregnant subject at less than 37 weeks gestation.

2. The method of claim 1, further comprising detecting a second biomarker complex of pre-B-cell colony-enhancing factor (PBEF), serum retinol binding protein (RBP4), apoliprotein C1 (ApoC1), interleukin 6 (IL-6), interleukin 8 (IL-8), relaxin, caspase recruitment domain containing protein CARD 12, or ferritin.

3. The method claim 1, further comprising detecting a serum retinol binding protein (RBP4), apoliprotein C1 (ApoC1), and pre-B-cell colony-enhancing factor (PBEF) biomarker complex.

4. The method of claim 1, further comprising detecting serum retinol binding protein (RBP4), apoliprotein C1 (ApoC1), pre-B-cell colony-enhancing factor (PBEF), and interleukin 6 (IL-6) biomarker complexes.

5. The method of claim 1, further comprising detecting serum retinol binding protein (RBP4), apoliprotein C1 (ApoC1), pre-B-cell colony-enhancing factor (PBEF), interleukin 6 (IL-6), and interleukin 8 (IL-8) biomarker complexes.

6. The method of claim 1, further comprising detecting serum retinol binding protein (RBP4), apoliprotein C1 (ApoC1), pre-B-cell colony-enhancing factor (PBEF), interleukin 6 (IL-6), interleukin 8 (IL-8), and relaxin biomarker complexes.

7. The method of claim 1, further comprising detecting serum retinol binding protein (RBP4), apoliprotein C1 (ApoC1), pre-B-cell colony-enhancing factor (PBEF), interleukin 6 (IL-6), interleukin 8 (IL-8), relaxin, and caspase recruitment domain containing protein CARD 12 biomarker complexes.

8. The method of claim 1, further comprising detecting serum retinol binding protein (RBP4), apoliprotein C1 (ApoC 1), pre-B-cell colony-enhancing factor (PBEF), interleukin 6 (IL-6), interleukin 8 (IL-8), relaxin, caspase recruitment domain containing protein CARD 12, and ferritin biomarker complexes.

9. A method for evaluating a pregnant patient comprising:
detecting one or more biomarker complexes that are indicative of labor in a sample from the patient, wherein a first biomarker is serum retinol binding protein (RBP4) or apolipoprotein C1 (ApoC1) and at least a second biomarker is pre-B-cell colony-enhancing factor (PBEF), serum retinol binding protein (RBP4), apolipoprotein C1 (ApoC1), interleukin 6 (IL-6), interleukin 8 (IL-8), relaxin, caspase recruitment domain containing protein CARD 12, and/or ferritin; and
determining if the patient is in labor or at risk of beginning preterm labor.

10. The method of claim 9, wherein the levels of ApoC1 and RBP4 are assessed.

11. The method of claim 9, wherein the levels of PBEF, ApoC1, and RBP4 are assessed.

12. The method of claim 9, wherein the levels of IL-6, PBEF, ApoC1, and RBP4 are assessed.

13. The method of claim 9, wherein the levels of RBP4, ApoC1, PBEF, IL-6, IL-8, relaxin, CARD 12, and ferritin are assessed.

14. The method of claim 9, wherein the sample is a blood, a cervical fluid, a urine, an amniotic fluid and/or a saliva sample.

15. The method of claim 9, wherein the patient is at risk of preterm labor or is suspected of being in preterm labor.

16. The method of claim 9, wherein the patient is less than 37 weeks pregnant.

17-20. (canceled)

21. The method of claim 9, wherein measuring the level of a biomarker comprises detecting protein and/or nucleic acid levels of the biomarker in the sample.

22-28. (canceled)

29. A method for monitoring the progression of preterm labor in a subject, the method comprising:
(a) detecting in a sample from the subject at a first time point, one or more biomarkers selected from serum retinol binding protein (RBP4) or apoliprotein C1 (ApoC1) and at least a second biomarker is pre-B-cell colony-enhancing factor (PBEF), serum retinol binding protein (RBP4), apoliprotein C1 (ApoC1), interleukin 6 (IL-6), interleukin 8 (IL-8), relaxin, caspase recruitment domain containing protein CARD 12, or ferritin;
(b) repeating step (a) at a subsequent point in time; and
(c) comparing levels detected in steps (a) and (b), and thereby monitoring the progression of preterm labor.

30-34. (canceled)
